(19) United States
(12) Patent Application Publication
(10) Pub. No.: US 2026/0306305 A1
(43) Pub. Date: Oct. 8, 2026

(54) COMPRESSION THERAPY ARRANGEMENT AND METHODS FOR OPERATING AND MONITORING THE SAME

(71) Applicant: Lohmann & Rauscher GmbH, Wien (AT)

(72) Inventor: Adam VAN ESSEN, Milwaukee, WI (US)

(21) Appl. No.: 19/717,521

(22) Filed: Jun. 23, 2026

Related U.S. Application Data
(62) Division of application No. 17/634,910, filed on Aug. 7, 2020.
(60) Provisional application No. 62/889,754, filed on Aug. 21, 2019.

(51) Int. Cl. A61F 13/08; A61F 13/00; A61F 13/06; A61H 7/00; G01L 1/14; G16H 40/67

(52) U.S. Cl. CPC A61F 13/08; A61F 13/06; A61H 7/001; G01L 1/142; G16H 40/67; A61F 2013/0028; A61H 2201/5074; A61H 2209/00

(57) ABSTRACT

The present invention relates to a compression therapy arrangement comprising a compression garment adapted to exert external pressure on a body portion, at least one sensor device associated with the compression garment and having at least one pressure sensor capable of producing at least one pressure signal indicative of the external pressure and transmitter means linked to the at least one sensor device and operable to transmit therapy signals corresponding to the pressure signals to an external device. The present invention also relates to methods for monitoring the compression therapy arrangement as well as a corresponding computer-readable medium and a corresponding computing system.

### CROSS REFERENCE TO RELATED APPLICATIONS

This patent application is a Divisional of U.S. patent application Ser. No. 17/634,910, filed Feb. 11, 2022 which is a U.S. National Phase Application under 35 U.S.C. § 371 of International Application No. PCT/US2020/045458, filed Aug. 7, 2020, entitled COMPRESSION THERAPY ARRANGEMENT AND METHODS FOR OPERATING AND MONITORING THE SAME, which claims the benefit of U.S. Provisional Application No. 62/889,754 filed on Aug. 21, 2019, the entire contents of which are hereby incorporated by reference in their entirety.

### TECHNICAL FIELD

This invention relates to a compression therapy arrangement, methods for operating and monitoring the same, a computer-readable medium, and a computing system.

### BACKGROUND

Compression therapy is for example applied in the treatment of edema which may be attributable to multiple pathologies. Two common but not exhaustive sources of edema in the lower extremities are chronic venous insufficiencies (CVI) and lymphedema. An approach for treatment and management of edema involves compression through appropriate compression garments, like bandages, hosiery, straps, stockings or the like. Success of therapy greatly depends on proper and repeated application of compression to the affected body portion. Further, long-term patient compliance/adherence of daily use at a specified conditions, such as duration and external pressure, is important for maintenance of chronic edema.

### SUMMARY

This invention is directed to providing a compression therapy arrangement and associated methods of operating and monitoring the same as well as a corresponding computer-readable medium and a corresponding computing system, which each help to improve compression therapy applied via a compression garment.

According to a general aspect of this invention, a compression therapy arrangement is provided which comprises at least one compression garment adapted to exert external pressure on a body portion, at least one sensor device associated with the compression garment and having at least one pressure sensor capable of producing at least one pressure signal indicative of the external pressure and transmitter means linked to the at least one sensor device and operable to transmit a therapy signal corresponding to the pressure signal to an external device. Embodiments of the present invention enable monitoring of the compression garment.

According to another general aspect of this invention, a method for operating the compression therapy arrangement is provided, wherein a therapy signal is transmitted to an external device, such as a mobile communication device.

According to another general aspect of this invention, the compression therapy arrangement is used for the treatment of edema.

According to another general aspect of this invention, a method comprises: using at least one computer hardware processor to perform the following operations: obtaining an identifier of a compression therapy arrangement, the compression therapy arrangement including a compression garment; determining an operational state of a portion of the compression garment based on the obtained identifier; and generating instructions for displaying of an indicator of the determined operational state. Embodiments of the present invention assist the user in properly operating the underlying technical system of the compression therapy arrangement, in particular the compression garment.

According to another general aspect of this invention, a method comprises: using at least one computer hardware processor for performing the following operations: displaying one or more compression therapy set-up procedure screens that require user input; and displaying one or more compression therapy treatment screens that graphically illustrate in at least substantially real-time measured values of at least one operational parameter associated with a compression therapy arrangement, the compression therapy arrangement including a compression garment being used for a compression therapy. Embodiments of the present invention allow tracking values of one or more operational parameters of the compression therapy arrangement in real-time or substantially real-time, which provides the user with continued feedback about the operation of the underlying technical system of the compression therapy arrangement, in particular the compression garment.

According to other general aspects of this invention, a computer-readable medium and a computing system corresponding to the methods of these general aspects are provided.

The at least one computer hardware processor of these methods may be located in the external device mentioned above that is external to the compression therapy arrangement, preferably wherein the external device is a mobile communication device. The operational state and/or the measured values of the at least one operational parameter may be obtained based on the therapy signals received by the external device from the compression therapy arrangement.

According to this invention, proper application of compression therapy may be monitored via the external device, e.g. a mobile communication device such as a smartphone, a tablet computer or the like, by using an appropriate pressure sensor which is associated with the compression garment and adequate transmitter means (sensor device) which on the one hand is linked to the at least one sensor device and on the other hand is capable of transmitting a therapy signal to an external device. Within the framework of this invention it is also contemplated to integrate transmitter means into the sensor device. In other embodiments of the invention at least one sensor of the sensor device is separated from the transmitter means in order to increase patient comfort.

According to the invention, the pressure signal produced by the sensor device may be an analogous signal, while the therapy signal which is derived from the pressure signal and transmitted to the external device may be a digital signal. In other embodiments of the invention pressure signal and therapy signal may be both digital signals or even identical signals. Further, a wireless transmission path may be established between the transmitter means and the external device to thereby avoid limitations caused by wired signal transmission. On the other hand, a pressure signal may be transmitted to the transmitter device by wire to thereby avoid excessive efforts for realizing pressure devices. The sensor device and the transmitter means may be attached to a compression garment where releasable attachment is preferred, e.g. via a hook and loop fastener where the hook portion of the fastener may be associated with the sensor device, while the loop portion of the fastener may be associated with the garment; or by use of a snap mechanism. In other embodiments of the invention at least one sensor and/or the transmitter means are permanently affixed to the garment. In order to arrive at an appropriate support of the user the external device may have processor means operable to process the therapy signal which processor means may be realized within a smartphone or another mobile communication device.

In a further preferred embodiment of the invention the proposed compression therapy arrangement further comprises converter means linked to said processor and operable to generate a perceptible signal corresponding to the external pressure. Those converter means may comprise display means linked to said processor means and operable to display a visual representation of said external pressure. Alternatively or additionally, said converter means may comprise speaker means capable of generating an audible signal corresponding to said external pressure and/or vibration generation means capable of generating perceptible vibrations. Alternatively or additionally, a sound sequence and/or vibration sequence corresponding to the progress of compression therapy with time and/or duration of compression therapy may be produced. Storage means may be provided which are capable of storing pressure data corresponding to a plurality of external pressure values representing a plurality of pressure signals which for example may be obtained successively in pre-determined time intervals, to produce a graphic representation of a graph illustrating the progress of compression therapy with time and/or the duration of compression therapy which may be represented by the length of the graph displayed on said display means. Graphic representation or an audible representation of the progress of compression therapy may help to increase patient adherence towards educated targets. Storage means may be associated with and/or integrated in said transmitter means and/or said processor means.

In a further preferred embodiment of the invention input means are provided which are linked to said processor means operable to input external data, e.g. user data, device data, target data, etc. which may be used for various purposes.

In a particularly preferred embodiment of the invention, said pressure sensor comprises at least one capacitor having a capacity which is dependent on the external pressure. This allows to determine high-precision external pressure determination in case the at least one capacitor is integrated into an electric circuit further comprising a resistance and/or inductance, such as a RC circuit or a LC circuit, and the pressure signal is determined based on a frequency analysis, such as to obtain the natural frequency or eigenfrequency of the LC circuit. The pressure sensor may have a flexible construction to ensure comfort and accuracy of measurement on uneven anatomical surfaces e.g. circumference of small limb. At least one pressure sensor and/or said transmitter means may comprise a flexible circuit board which is integrated into or attached to the garment.

In a further preferred embodiment of the invention said sensor device additionally comprises at least one of a temperature sensor, a moisture sensor, a bioimpedance sensor system, a movement sensor, an Inertial Measurement Unit that measures movement and activity, an acceleration sensor and a position sensor to thereby open the possibility to monitor progress of therapy and the circumstances of therapy in more detail.

In case additional sensors are provided, said transmitter means is preferably operable to transmit to an external device at least one of a temperature signal derived from said temperature sensor, a movement signal derived from said movement sensor, an acceleration signal derived from said acceleration sensor and a position sensor derived from said position sensor. Also these additional signals are preferably transmitted to the external device via a wireless communication channel, optionally also as digital data.

In order to avoid an unauthorized or otherwise undesired link of said processor means which may be embodied by a smartphone or other mobile communication device to undesired compression therapy arrangements, in a preferred embodiment of the invention said operator means is operable to compare input data to device data indicative of said sensor device and/or said transmitter means, transmitted by said transmitter means. It may be further operable to enable operation of said processor means for controlling said display means to display graphic representations of any of the signals received from said transmitter means and derived from signals of the sensor device depending on the result of the comparison. Thus, in this preferred embodiment of the invention, only in case an appropriate verification process by comparing input device data to device data received from the compression therapy arrangement is successfully finalized the operation of the processor means in combination with specific compression therapy arrangement is allowed. In other words, only in the case where device information is sent with input data, i.e. the processor means is paired with the device, the processor may be successfully used. Obviously it is also contemplated within the framework of this invention to pare processor means with two or more compression therapy arrangements. In this case the user may be asked to select a specific garment via said input means. It may be tracked, which garment is being used and how long specific garments are used.

The above mentioned device data received from the compression therapy arrangement may be encoded in a code, such as a QR code or a bar code. The compression therapy arrangement may comprise the code, wherein the code encodes an identifier of the compression garment and wherein the code is scannable such that the identifier is obtainable through scanning of the code. The code may be attached to the compression garment or to a package thereof.

Use of a specific processing means may further depend on successful input and comparison of other user data, e.g. e-mail address, user name, and password.

Further, use of the processing means may be blocked in case the user does not acknowledge contraindications of the compression device by appropriate input upon respective request.

In a further preferred embodiment of the invention, the sensor device may visually indicate if the external compression is within, above or below a user defined target range i.e. RGB LED with yellow for below range, green within range, and red above range of 20-30 mmHg). This may further help to increase patient adherence to educated targets.

In a further preferred embodiment of the invention, said processor means may be operable to compare input target data with progress data derived from said stored pressure data. Said target data may be input for day and night, for specific time segments, for target activity level and depending on compression garment type (i.e. knit, chopped foam, wrap, bandaging). The comparison between input target data and progress data may also allow to enable display of specific items. For this purpose, processor means may be operable to enable processor functions, e.g. control of display means to display specific items depending on the result of the comparison between said target input data and said progress data. In this way, elements of gamification showing statistics in a nice and appropriately sized shape at the top of the display may be produced. Below statistics derived from the progress data locked badges may be displayed where it should be made sure that it is obvious that these badges are inaccessible. This may entice the user to unlock them every time they visit the application by appropriate use of the processor means and associated compression therapy arrangements.

Operation of the proposed compression therapy arrangement may be embedded into social networks. For this purpose, the processor means may be operable to transmit pressure data, user data, device data and/or target data to an external web server for sharing such data and information generated on the basis thereof between a community of users. For the same purpose, the processor means of the proposed compression therapy arrangement may be operable to receive web data from an external web server and to control said display means to display a graphic representation of said web data, e.g. together with a graphic representation of said pressure data and/or said external data. In this embodiment of the invention public profiles may be generated and discussion forums may be established. Further, said web data may contain educational content and reference information, links, occasional pop-up quick tips etc., which content may be associated with main processor means and not directly associated with the community.

In a further preferred embodiment, said processor means may determine garment life status. For this purpose, the processor means may evaluate pressure data, user data and device data to determine garment life based on parameters of wash cycles, usage durations, and other user factors. Garment life may then be graphically displayed.

In a further preferred embodiment, said processor means may be operable to receive usage data indicative of the period of usage of said compression garment and to compare said usage data to respective target data to thereby produce graphic representations indicative of the end of life of the garment, alert data in case compression is out of range, device out of range/not connected, reminder to wash garment, reminder to submit daily log entry or to do daily activity, etc. Under the aspect of data security it is preferred that this processor means is operable to transmit data to one or more external hosts wherein said processor means may be operable to divide and/or encrypt data for transmission to said at least one external host and to share data between multiple encrypted tables (e.g. patient profile table, device registration table, recorded data table). Such data may be accessible at least in part by customer service for telephone support and as appropriate data analysis tool.

In all embodiments of the invention, said compression garment may comprise at least one compression sleeve, and/or at least one compression bandage and/or at least one preferably adjustable compression strap. Examples of appropriate compression garments are shown for example in U.S. Pat. No. 8,221,340 B2. The content thereof with respect to the construction of compression garments is incorporated herein by reference.

Exemplary embodiments of the invention are shown in the drawing wherein like reference numerals are referred to identical or equivalent features.

### BRIEF DESCRIPTION OF THE DRAWINGS

FIG. 1 illustrates a schematic representation of an exemplary embodiment of a compression therapy arrangement according to the invention.

FIG. 2 illustrates a schematic representation of a second exemplary embodiment of a compression therapy arrangement according to the invention,

FIG. 3 shows the arrangement according to FIG. 1 in actual use, and

FIG. 4 shows the arrangement according to FIG. 2 in actual use.

FIGS. 5, 6 and 9 show illustrating graphical outputs by the external device, being in communication with the compression therapy arrangement, for monitoring the operation of the compression therapy arrangement, such as the compression therapy arrangement according to FIG. 1 or FIG. 2. FIGS. 5, 6 and 9 also show optional means for user input via the external device, the user input to be used for the monitoring of the operation of the compression therapy arrangement.

FIGS. 7 and 8 show further optional means at the graphical user interface of the external device for user input to be used for the monitoring of the operation of the compression therapy arrangement as shown by FIGS. 3, 4, 5, 6 and 9.

FIG. 10 illustrates a networked environment for the compression therapy arrangement.

### DETAILED DESCRIPTION

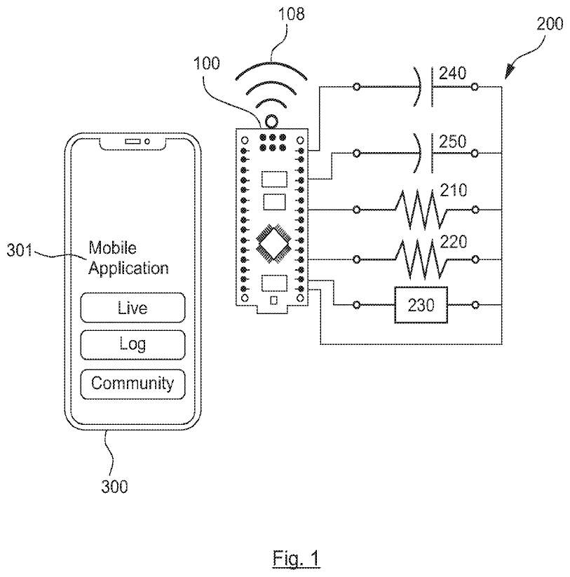

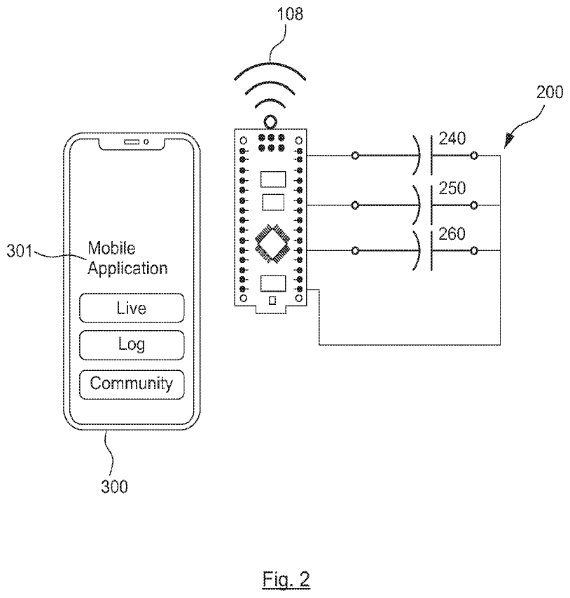

The compression therapy arrangement of each of FIG. 1 and FIG. 2 comprises a compression garment 10 adapted to exert external pressure on a body portion 12, at least one sensor device 200 associated with the compression garment 10 and having at least one pressure sensor 240, 250, 260 capable of producing at least one pressure signal indicative of the external pressure and transmitter means 100 linked to the at least one sensor device 200 and operable to transmit therapy signals 108 corresponding to the pressure signals to an external device 300.

The compression therapy arrangement shown in FIG. 1 comprises the sensor device 200 and the transmitter means 100 linked to the sensor device and to the external device 300, such as a mobile communication device, which may be realized by a smartphone and usable as graphic user interface for the compression therapy arrangement. The sensor device 200 of FIG. 1 may exemplary comprise one or more resistive sensors 210, 220 and/or one or more sensors 230 which are mainly based on additional or other physical processes such as inductive sensors, each linked by wire to the transmitter means and one or more capacitive pressure sensors 240 and 250, also linked by wire to the transmitter means 100. Sensor 210 may be a temperature sensor, sensor 220 may be a moisture sensor, and sensor 230 may be a bioimpedance sensor system, a movement sensor, an acceleration sensor or a position sensor.

Further to comprising transmitting elements, transmitter means 100 may comprise additional circuitry for deriving temperature and/or pressures signals from sensor device 200, e.g. inductive elements and frequency analyzer elements for deriving pressure value from pressure dependent values, such as capacity values of capacitors 240 and 250.

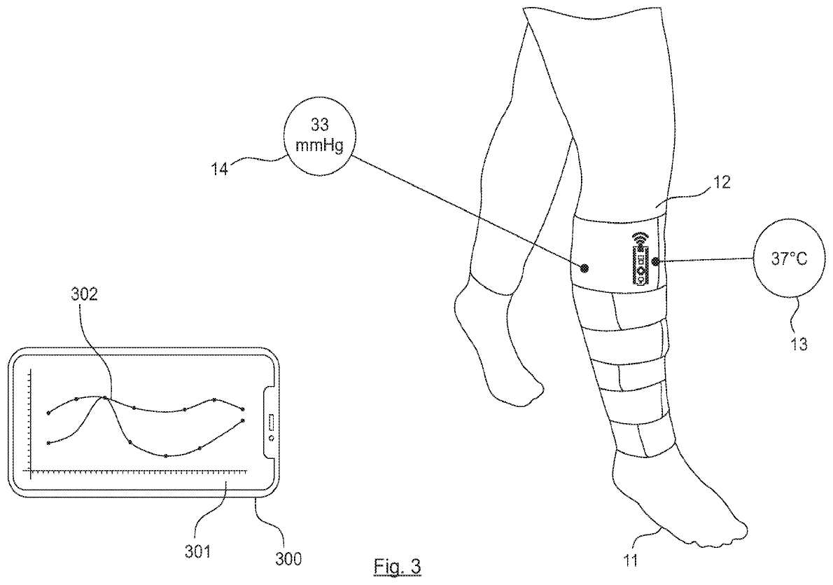

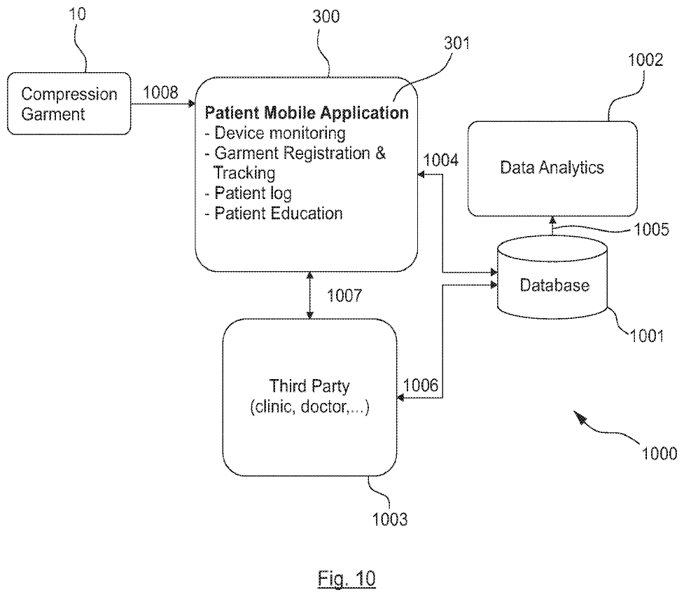

As shown in FIG. 1 the external device 300 comprises converter means realized by a display which display may also be used as input means for inputting user data and user commands. The external device 300 may also be used as a web client communicating with an external entity, such as third party component 1003 that may be a server, web interface and/or another mobile application, or such as database 1001 of FIG. 10. The external device 300 may have installed thereon an application 301, such as a mobile application (app), which is capable to generating an indicator, such as indicator 302 in FIG. 3, providing a graphic representation of pressure-related data, such as in form of one or more graphs illustrating development of current pressure 14 and/or temperature 13 level.

The embodiment shown in FIG. 2 mainly differs from the embodiment illustrated in FIG. 1 in that it comprises a sensor device 200 having three capacity pressure sensors 240, 250 and 260, each linked by wire to transmitter means 100.

According to FIG. 2 temperature sensor 210 (not shown) and three highly flexible pressure sensors 240, 250, 260 are attached to or integrated into a compression garment or bandage. Temperature and pressure signals are transmitted via transmitter means to external device 300 which is capable of storing a plurality of temperature and/or pressure data and generating a graphic representation of pressure data and external data as a graph illustrating development of pressure levels 14, 15, 16 and/or temperature level 13.

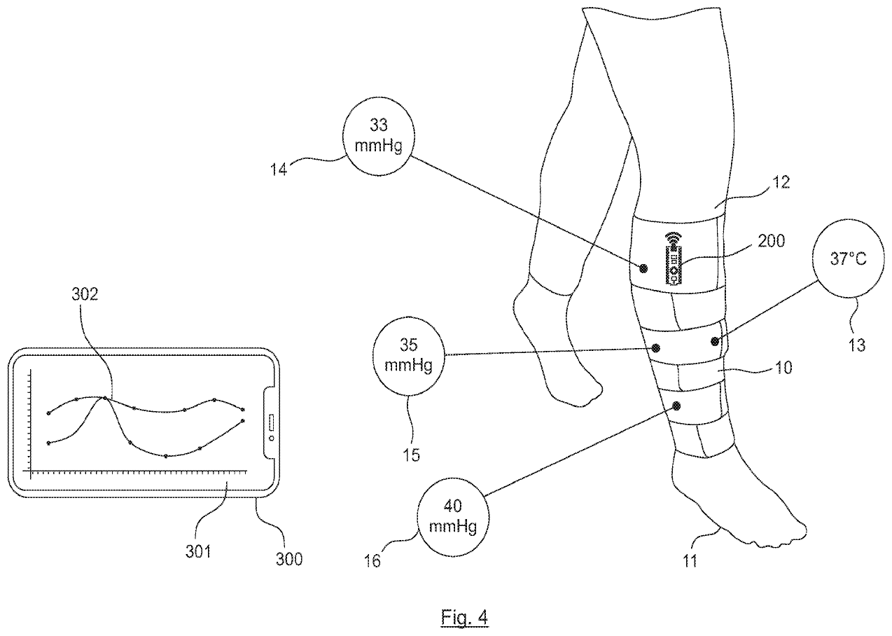

According to FIG. 4 the capacitive pressure sensors 240, 250 and 260 of the arrangement shown in FIG. 2 are attached to a compression bandage 10 such that a first pressure sensor 240 is arranged at a proximal end portion of the bandage 10, a second pressure sensor 250 is arranged at a central portion of bandage 10 and a pressures sensor 260 is arranged at a distal end portion of the bandage 10. Further, temperature sensor which is not shown in FIG. 2 is provided. Sensor signals from each of said sensors 210, 240, 250 and 260 are transmitted via transmitter means 100 to the external device 300 which has installed thereon an app 301 which is capable to generating an indicator 302 providing a graphic representation of pressure data 14, 15, 16 and temperature data 13, and optionally external data as one or more graphs 302 illustrating development of pressure 14, 15, 16 and/or temperature 13 level.

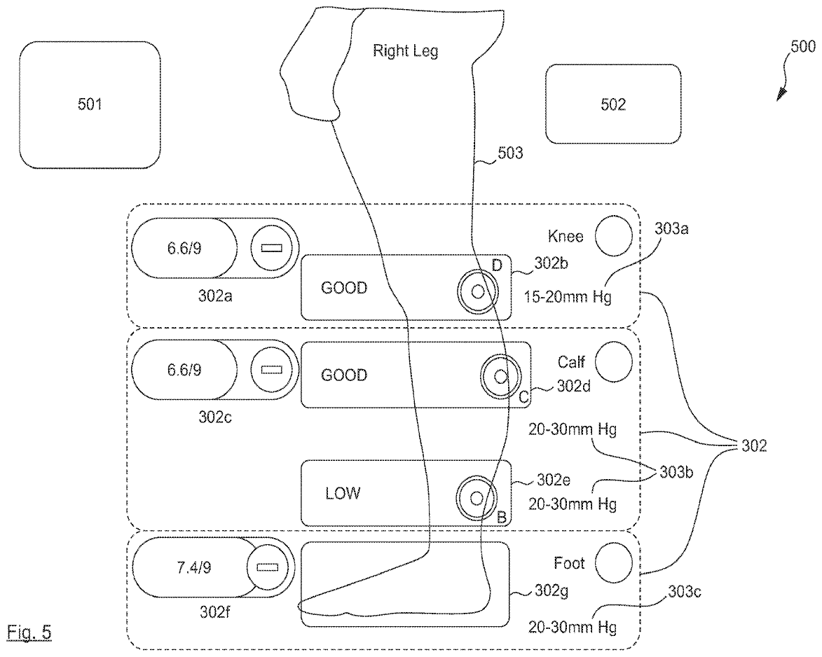

FIGS. 5, 6 and 9 show illustrating graphical outputs by the external device being in communication with the compression therapy arrangement for monitoring the operation of the compression therapy arrangement, such as the compression therapy arrangement according to FIG. 1 on 2. FIGS. 5, 6 and 9 also show optional means for user input via the external device, the user input to be used for monitoring of the operation of the compression therapy arrangement.

In particular, FIGS. 5, 6 and 9 show graphical outputs 500, 600, 900 via a graphical user interface of the external device 300, wherein the graphical output 500, 600, 900 may be generated by the application 301 and may be displayed by the display of the external device 300.

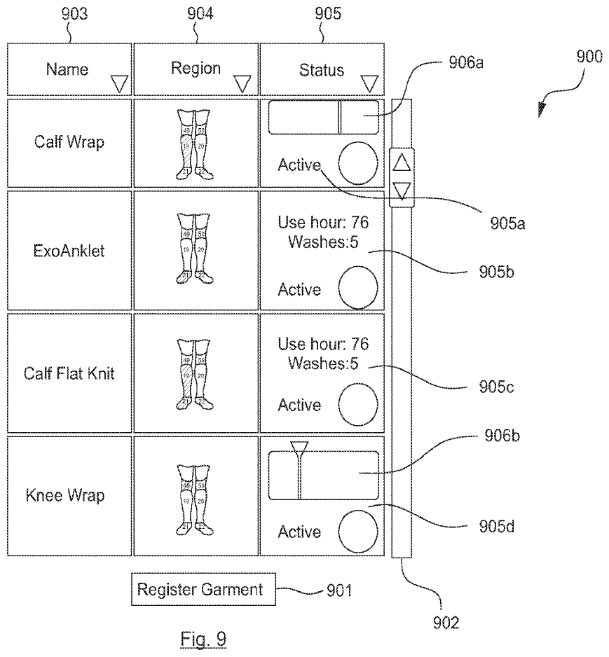

FIGS. 5 and 9 illustrate exemplary indicators 302, 302a, 302b, 302c, 302d, 302e, 302f, 302g, 302h, 302i, 905a, 905b, 905c, 905d, 906a, 906b indicating operational states of the compression garment 10. The operational states referred to in this disclosure may dynamically change and may be determined automatically. The corresponding indicators 302, 302a, 302b, 302c, 302d, 302e, 302f, 302g, 302h, 302i, 905a, 905b, 905c, 905d, 906a, 906b may be presented and updated in real-time or in substantially real-time. Alternatively, these indicators may not be presented and updated in real-time or in substantially real-time, but rather are presented and updated at particular times during the course of the running application 301, such as when the application 301 is started and/or at periodic intervals between connecting of the application 301 with the compression therapy arrangement and disconnecting of the application 301 with the compression therapy arrangement.

At least one computer hardware processor located in the external device 300 is configured to perform the following operations: obtaining an identifier of a compression therapy arrangement, the compression therapy arrangement including a compression garment 10; determining an operational state of a portion of the compression garment 10 based on the obtained identifier; and generating instructions for displaying of an indicator 302, 302a, 302b, 302c, 302d, 302e, 302f, 302g, 302h, 302i, 905a, 905b, 905c, 905d, 906a, 906b of the determined operational state. The display of the external device 300 may display the indicator 302, 302a, 302b, 302c, 302d, 302e, 302f, 302g, 302h, 302i, 905a, 905b, 905c, 905d, 906a, 906b of the determined operational state. The operational state is determined by using the therapy signals 108 transmitted by the transmitter means 100 and received by the external device 300.

The external device 300 may be configured to scan a code, wherein the code encodes the identifier of the compression therapy arrangement and wherein the identifier is obtained through the scanning of the code. The code may be a QR code or a bar code.

Based on the obtained identifier, the external device 300 can obtain, from a local or remote data store, such as remote database 1001 described in context of FIG. 10, at least one range of allowable and/or targeted values 303a, 303b, 303c for at least one operational parameter of the portion of the compression garment 10, wherein the indicator 302, 302a, 302b, 302c, 302d, 302e, 302f, 302g, 302h, 302i, 905a, 905b, 905c, 905d, 906a, 906b may indicate the obtained at least one range of allowable and/or targeted values 303a, 303b, 303c. In other words, based on the obtained identifier of the compression garment 10, the external device 300, in particular the application 301, can obtain identification of which type of compression garment is to be used (paired) with the external device 300 and the application 301 can implement the boundaries of proper operation of the particular garment 10 being used (paired) with the external device 300 in the application 301, to prevent unapproved operation of the garment 10 by the user (patient).

The determining of the operational state may include obtaining, by the external device 300 and from the sensor device 200 via the transmitter means 100, one or more measured values 302h for the at least one operational parameter of the portion of the compression garment 10.

The determining of the operational state may include comparing the one or more measured values 302h with the at least one range of allowable and/or targeted values 303a, 303b, 303c, wherein the indicator 302, 302a, 302b, 302c, 302d, 302e, 302f, 302g, 302h, 302i, 905a, 905b, 905c, 905d, 906a, 906b may indicate a result of the comparing of the one or more measured values 302h with the at least one range of allowable and/or targeted values 303a, 303b, 303c.

The indicator 302, 302a, 302b, 302c, 302d, 302e, 302f, 302g, 302h, 302i, 905a, 905b, 905c, 905d, 906a, 906b may indicate how the measured values 302h relate to the range of allowable and/or targeted values 303a, 303b, 303c.

The portion of the compression garment 10 may include a plurality of subparts, such as subparts “B”, “C” and “D”, of the compression garment 10, the at least one range of allowable and/or targeted values 303a, 303b, 303c includes a plurality of ranges of allowable and/or targeted values 303a, 303b, 303c for the at least one operational parameter with each range of the plurality of ranges 303a, 303b, 303c being associated with a corresponding subpart of the plurality of subparts, and wherein the indicator 302, 302a, 302b, 302c, 302d, 302e, 302f, 302g, 302h, 302i, 905a, 905b, 905c, 905d, 906a, 906b indicates, for each subpart of at least some of the plurality of subparts of the compression garment 10, how respective ones of the measured values measured for the respective subpart relate to an associated one of the ranges of allowable and/or targeted values 303a, 303b, 303c corresponding to the subpart of the plurality of subparts.

As illustrated in an exemplary manner in FIG. 5, indicator 302b indicates that the pressure exerted by subpart D of the compression garment 10 being applied to the knee of the right leg of the patient is within the range of allowable pressure values 303a associated with this subpart D of the compression garment at the knee. Indicator 302d indicates that the pressure exerted by subpart C of the compression garment 10 being applied to the calf of the right leg of the patient is within the range of allowable pressure values 303b associated with this subpart C of the compression garment at the calf Indicator 302e indicates that the pressure exerted by another subpart B of the compression garment 10 being applied to the calf of the right leg of the patient is below the range of allowable pressure values 303b associated with this subpart B of the compression garment at the calf.

As also illustrated in an exemplary manner in FIG. 5, indicator 302a indicates that the operating time or wearing time of the subpart D of the compression garment 10 being applied to the knee of the right leg of the patient is within the range of targeted values associated with this subpart D of the compression garment at the knee and is 6.6 hours out of targeted 9 hours. Indicator 302c indicates that the operating time or wearing time of the subpart C of the compression garment 10 being applied to the calf of the right leg of the patient is within the range of targeted values associated with this subpart C of the compression garment at the calf and is here also 6.6 hours out of targeted 9 hours. Indicator 302f indicates that the operating time or wearing time of a subpart of the compression garment 10 being applied to the foot of the right leg of the patient is within the range of targeted values associated with this subpart of the compression garment at the foot and is here 7.4 hours out of targeted 9 hours.

As illustrated by indicator 302g, the operational state may include an operational state of a particular subpart (subpart “A” at the foot, not shown) of the plurality of subparts, wherein the indicator 302, 302g indicates that the particular subpart is not yet activated for read-out of the at least one operational parameter. The reason may be that the particular subpart of the compression garment 10 does not yet have a sensor for measuring values of the at least one operational parameter, such as a pressure sensor 240, 250, 260 for measuring values of exerted pressure, or that the sensor is present at the particular subpart of the compression garment 10 but the sensor is not activated for read-out of values of the at least one operational parameter. In other words, indicator 302g pertains to an operational state of inactivity (hence subpart “A” at the foot is not shown) of the monitoring of the subpart of the compression garment 10 being applied to the foot of the right leg of the patient. The operations performed by the hardware processor of the external device may further comprise: receiving an activation signal for activating read-out of the at least one operational parameter for the particular subpart (e.g., subpart “A” at the foot); and in response to the receiving of the activation signal, changing the indicator 302g of the operational state of the particular subpart such that the changed indicator 302g indicates that the particular subpart is activated for read-out of the at least one operational parameter.

Each of the ranges of allowable and/or targeted values may form a part of the corresponding indicator 302, 302a, 302b, 302c, 302d, 302e, 302f, 302g or may be separate therefrom.

The indicator 302, 302a, 302b, 302c, 302d, 302e, 302f, 302g, 302h, 302i, 905a, 905b, 905c, 905d, 906a, 906b may include a graphical illustration 503, 603, 703, 803 of a portion of a human body, wherein the graphical illustration is also graphically outputted via the graphical user interface of the external device 300. The indicator 302, 302a, 302b, 302c, 302d, 302e, 302f, 302g, 302h, 302i, 905a, 905b, 905c, 905d, 906a, 906b may indicate how the measured values 302h relate to the range of allowable and/or targeted values 303a, 303b, 303c at a location of the graphical user interface that is associated with a subpart of the graphical illustration corresponding to a body portion (e.g. body part) at which the corresponding portion of the compression garment 10 is applied to the patient.

For example, as illustrated in FIG. 5, each of the indicators 302a-302g are graphically outputted/displayed via the graphical user interface of the external device 300 and in reference to or in association with the subpart of the graphical illustration 503, 603, 703, 803 corresponding to the body portion at which the subpart (here: A, B, C, D) of the compression garment 10 is applied to the patient. For example, indicator 302b relates to a knee, as explained above, and is graphically outputted/displayed above a graphical illustration of the knee of the right leg of the patient. However, indicator 302b may also be graphically outputted/displayed in a manner not overlapping (e.g. disjoint) with the graphical illustration of the knee, but still at a location of the graphical user interface that is associated with the graphical illustration of the knee of the right leg of the patient at which the corresponding subpart D of the compression garment 10 is applied to the patient. For example, indicator 302b may be graphically outputted/displayed in a manner not overlapping (e.g. disjoint) with the graphical illustration of the knee, but with a displayed graphical link between the indicator 302b and the graphical illustration of the knee.

FIG. 5 also shows optional means 501, 502 at the graphical user interface of the external device 300 for user input to be used for the monitoring of the operation of the compression therapy arrangement. For example, the graphical user interface may display an element 501 configured to receive user input, such as user input causing activation of a (daily) log of the operational states. The graphical user interface may also display an element 502 being configured to receive another user input, such as user input causing an adding of an additional compression therapy arrangement to be monitored.

The hardware processor of the external device 300 may further perform the operations comprising: receiving, for a subpart of the plurality of subparts or the portion of compression garment 10 and via the graphical user interface of the external device 300, user input pertaining to a change of a setting corresponding to the at least one operational parameter; and in response to the receiving of the change of the setting, generating instructions for display, via the graphical user interface, of a feedback message indicating whether or not the received change of the setting is in accordance with the at least one range of allowable and/or targeted values 303a, 303b, 303c that is associated with the subpart of the portion of the compression garment 10.

As illustrated in an exemplary manner in FIG. 5, embodiments of the present invention assist the user in properly operating the underlying technical system, namely the compression therapy arrangement.

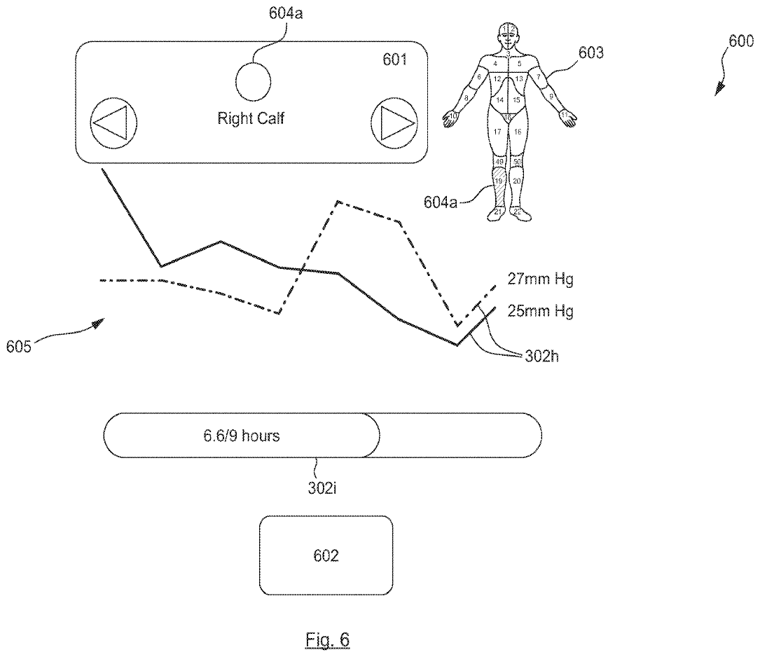

FIG. 6 illustrates graphical output 600 pertaining to a real-time or substantially real-time monitoring of measured values of an operational parameter of the compression therapy arrangement.

At least one computer hardware processor located in the external device 300 is configured to perform the following operations: displaying one or more compression therapy set-up procedure screens 601, 602 (see also 501, 502, 700, 800) that require user input; and displaying one or more compression therapy treatment screens 605 (see also 302, 905) that graphically illustrate in at least substantially real-time measured values 302h of at least one operational parameter associated with a compression therapy arrangement, the compression therapy arrangement including a compression garment 10 being used for a compression therapy. The measured values 302h of at least one operational parameter are obtained from the therapy signals 108 transmitted by the transmitter means 100 and received by the external device 300.

The operational parameter being monitored in real-time or in substantially real-time may be an operational parameter of the compression garment 10, such as an exerted pressure measured by one of pressure sensors 240, 250, 260. As also illustrated by the solid and dashed graphs in indicator 302h, values measured at multiple sensors pertaining to the same or multiple different operational parameters may be displayed via the graphical user interface in a compression therapy treatment screens 605. In FIG. 6, the multiple graphs 302h pertain to multiple different pressure sensors, such as the pressure sensors associated with subparts B and C in FIG. 5. The graphical output 600 or the one or more compression therapy treatment screens 302, 605, 905 may preferably graphically illustrate the measured values of the at least one operational parameter in association with a particular body portion 12, 604a, 704 to which the compression garment 10 is attached. That is, the relevant body portion 604a where the one or more operational parameters are being monitored in real-time or in substantially real-time is preferably indicated.

As illustrated in FIG. 6, the graphical output 600 or the compression therapy treatment screen 605 may also include another indicator 302i, in which the current operating time or wearing time in relation to a corresponding allowable value or target value is displayed, wherein in case of Figure 6.6 hours current operating time or wearing time out of 9 max. targeted hours are shown.

The one or more compression therapy treatment screens 302, 605, 905 may thus graphically illustrate the progress of at least one step in the compression therapy in at least substantially real-time.

The one or more compression therapy treatment screens 302, 605, 905 may graphically illustrate (see, e.g., indicator 302i) the measured values of the operational parameter in relation to a range of allowable and/or targeted values 303a, 303b, 303c for the operational parameter.

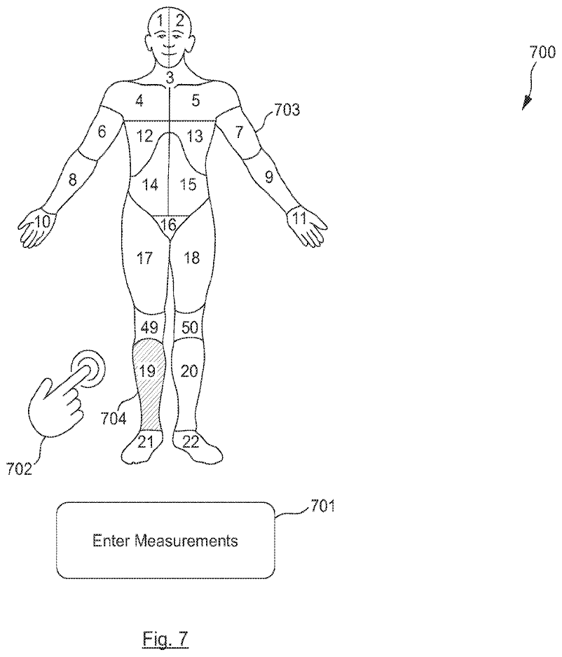

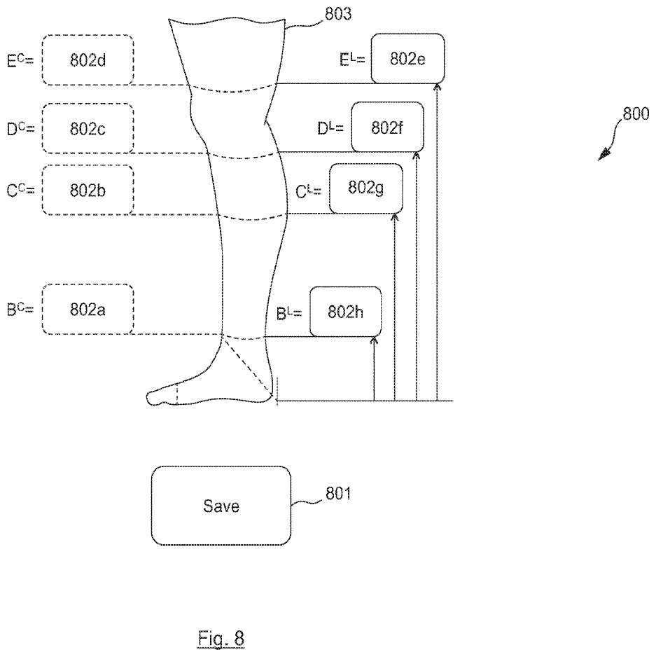

As shown in FIG. 6, the one more compression therapy set-up procedure screens 501, 502, 601, 602, 700, 800 may require user input pertaining to a body portion 12, 604a, 704 to which the compression garment 10 is attached. For example, the user of the external device 300 may switch between different body portions where subparts of the compression garment 10 are attached to obtain the corresponding real-time or substantially real-time measured values of the one or more operational parameters. In particular, the user may switch selection between the different body portions via the compression therapy set-up procedure screens 601 to obtain the respective current pressure values of the subpart of the compression garment attached to the selected body portion. The one or more compression therapy set-up procedure screens 501, 502, 601, 602, 700, 800 may also require user input pertaining to one or more dimensions of one or more body portions 12, 604a, 704 of a patient, a start and/or end time of a wearing time of the compression garment 10 by a patient, wash cycles applied to the compression garment 10 and/or a value of an external pressure exerted by the compression garment 10 onto a body portion 12, 604a, 704 of a patient, as also illustrated in FIGS. 7 and 8. Through such user input, the application 301 can also be used to track compression garment usage, such as the operating time or the wearing time as illustrated in an exemplary manner by indicators 302a, 302c and 302f in FIG. 5 or indicators 905b, 905c in FIG. 9, for compression therapy arrangements having no sensor device 200 that is in communication with the external device 300.

FIG. 6 also shows that graphical output 600, in particular screen 601, may include other graphical elements as needed, such as an element by which the user can also submit other user input, such as submit to stop the monitoring.

As illustrated in an exemplary manner in FIG. 6, embodiments of the present invention allow tracking values of one or more operational parameters of the compression therapy arrangement in real-time or substantially real-time, which provides the user with continued feedback about the operation of the underlying technical system, namely the compression therapy arrangement.

FIGS. 7 and 8 also show optional means at the external device for user input to be used for the monitoring of the operation of the compression therapy arrangement as shown by FIGS. 5, 6 and 9.

As illustrated by FIG. 7, the graphical user interface of the external device 300 may display a graphical illustration 703 of a human body. The graphical illustration 703 indicates individual ones of a plurality of body portions, which are selectable by the user, such as the patient. Upon selection by the user, the selected body portion 704 is displayed in a manner that highlights the selected body portion 704 to distinguish the selected body portion 704 from the other non-selected body portions. In this disclosure, a body portion may include a body part of the human body, may be a subpart of a body part of the human body or may be a body part of the human body.

As also illustrated by FIG. 7, the graphical user interface of the external device 300 may display an element 701, such as an icon, which the user can activate to enter measurements of body portions, such as selected body portion 704. The user may use one or more of his fingers 702 or another position indicator, such as a stylus, to perform this activation of the element 701 before or after selecting the body portion 704 for which the measurement is to be entered. A graphical output 800 with a graphical illustration 803 of a portion of the human body, as illustrated in FIG. 8, may be triggered in response to activating element 701 in which the user may enter, into graphically depicted fields 802a-802h of the graphical user interface of the external device 300, individual values for sizes of individual subparts of the selected body portion 704 in FIG. 7. The graphical output 800 may also include a graphical element 801 to save the entered sizes. The size may pertain to height, width, length and/or circumference of the respective body portion.

As discussed above in relation to FIGS. 5 and 6, the at least one operational state or operational parameter being monitored may, for example, include an external pressure exerted by the compression garment 10 on a body portion 12, 604a, 704 of a patient, availability of a sensor being activated for read-out out, and/or an operating time or wearing time of the portion of the compression garment 10. As illustrated in FIG. 9, the at least one operational state or operational parameter being monitored may additionally or alternatively include a number of washing cycles (washes) the compression garment 10 has experienced.

FIG. 9 illustrates, in an exemplary tabular form, the user specified identifier or names 903 of the compression garment, graphical illustrations of the regions 904 of the corresponding subparts of the compression garment 10 along the human body, and the corresponding operational state or status 905 of the corresponding subpart of the compression garment 10. The operational state or status 905 may include a remaining usable garment life 906a, 906b, whether or not a sensor is activated for read-out 905a, and operating times/wearing times as well as experienced washing cycles (washes) in indicators 905b-905d. The user may be enabled by the graphical user interface to scroll or switch between the different compression garments via graphical element 902. The graphical output 900 may further include other optional graphical elements, such as graphical element 901 to register an additional compression garment, which may involve scanning the code encoding the identifier of the additional compression garment.

A remaining usable garment life may be determined by the external device 300, and displayed graphically via its graphical user interface or display, such as by indicators 906a, 906b in FIG. 9, based on user input data and/or collected usage data. The collected usage data may include the operating time or wearing time of the compression garment 10 and the number of washing cycles the compression garment 10 has experienced, as shown in indicators 905b and 905c. That is, the remaining usable garment life may be determined or calculated as a function depending on the operating time or wearing time as well as on the number of washing cycles (washes). For example, indicator 906a may indicate that the corresponding subpart of the compression garment 10 has reached 70% of its possible garment life, leaving 30% of the garment life still available for usage. For example, indicator 906b may indicate that the corresponding subpart of the compression garment 10 has reached 20% of its possible garment life, leaving still 80% of the garment life available for usage.

The operations performed by the hardware processor of the external device may further comprise: generating instructions for display of guiding information, the guiding information including instructions for a user how to properly operate the compression garment 10.

FIG. 10 illustrates a networked environment 1000 and which the compression therapy arrangement including the garment 10 is communicating with the external device 300 via a communication link 1008, such as via Bluetooth or NFC, to transmit the therapy signals 108. Also, the external device 300 is communicating with a database 1001 via a communication link 1004, such as via a network connection (e.g., LAN, WAN, or Internet), and with a third party component 1003 via the communication link 1007, such as via a network connection (e.g., LAN, WAN, or Internet). The third party component 1003 may be a software component, a hardware component or a combination of both. In particular, the third party component 1003 may be a (web) server, web interface and/or another mobile application. The third party component 1003 is operated by the third party, such as clinic personnel or a physician.

For example, the above at least one range of allowable and/or targeted values 303a, 303b, 303c may be obtained by the external device 300 from the database 1001 via the communication link 1004.

The operations performed by the hardware processor of the external device may further comprise: transmitting, for each subpart of the plurality of subparts of the compression garment 10 or for the portion of the compression garment 10, the corresponding measured values 302h and associated timestamps to the database 1001 for storage in the database 1001.

The operations performed by the hardware processor of the external device 300 may further comprise: receiving, from the database 1001, based on historical values stored in the database 1001 wherein the historical values include values previously measured for the at least one operational parameter and associated historical timestamps, instructions for displaying recommendation information to a user operating the external device 300, the recommendation information including behavioral recommendations for the user to comply with a particular compression therapy and/or recommendations regarding proper usage of the compression garment 10.

The third party component 1003 may also be communicating with the database 1001 via communication link 1006, such as via a network connection (e.g., LAN, WAN, or Internet). Each of the communication links 1004, 1006, 1007 and 1008 may be a bidirectional communication link allowing for bidirectional communication between the linked entities. The communication link 1008 between the compression therapy arrangement, including the compression garment 10, and the external device 300 may also be one-directional such that the compression therapy arrangement may transmit signals, such as the therapy signal 108, to the external device 300, but not vice versa.

The database 1001 may be located in the memory of the external device 300 or may be remote to the external device 300. The database 1001 may be located in the memory of the third party component or may be remote to the third party component 1003. The database 1001 may have built-in functionality to perform data analytics 1002 for any one of the determinations or calculations described herein. The built-in functionality to perform data analytics 1002 may be employed by the external device 300 and/or the third party component 1003 to determine operational states of the compression garment and/or to determine guidance for the patient how to behave and/or properly operate the garment 10 to achieve a desired therapeutic result.

When the external device 300 is operated by the patient, the third party component 1003 may be operated by clinic personnel or a physician. That is, the patient may use the mobile communication device 300 throughout her/his daily routine to operate and/or monitor the compression therapy arrangement including the compression garment 10 and can transmit compression therapy-related data to the clinic personnel over the physician via communication link or network connection 1007.

For example, the operations performed by the hardware processor of the external device 300 may further comprise: storing the obtained measured values for the operation parameters on the external device and/or transmitting, via link or connection 1007, the obtained measured values to a pre-approved third party's server 1003.

The clinic personnel or the physician having access to the third party's server 1003 can then, based on the received therapy-related data, assess whether or not the compression therapy works properly or if the patient should receive any additional behavior information by the clinic personnel or the physician in context of medical advice. The operations performed by the hardware processor of the external device may further comprise: generating instructions for display of the behavior information, the behavior information including instructions for the patient how to behave to comply with a particular desired compression therapy.

The external device 300 in the environment 1000 of FIG. 10 provides via its application 301 any combination of the above described functionalities, such as device monitoring, compression garment registration and tracking, patient log and/or patient guidance/education.

Having described several aspects of at least one embodiment of this invention, it is to be appreciated that various alterations, modifications, and improvements will readily occur to those skilled in the art.

Such alterations, modifications, and improvements are intended to be part of this disclosure, and are intended to be within the scope of the invention. Further, though advantages of the present invention are indicated, it should be appreciated that not every embodiment of the technology described herein will include every described advantage. Some embodiments may not implement any features described as advantageous herein and in some instances one or more of the described features may be implemented to achieve further embodiments. Accordingly, this description and these drawings are by way of example only.

The above-described embodiments of the technology described herein can be implemented in any of numerous ways. For example, the embodiments may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. However, a processor may be implemented using circuitry in any suitable format.

Further, it should be appreciated that a computer, such as external device 300 or third party component 1003, may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable mobile or fixed communication device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the present invention may be embodied as a computer-readable storage medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments of the invention discussed above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the present invention as discussed above. As used herein, the term “computer-readable storage medium” encompasses only a non-transitory computer-readable medium that can be considered to be a manufacture (i.e., article of manufacture) or a machine. Alternatively or additionally, the invention may be embodied as a computer readable medium other than a computer-readable storage medium, such as a propagating signal.

The terms “application”, “app”, “algorithm”, “program code”, “computer program” or “software” are used herein in a generic sense to refer to any type of computer code or set of computer-executable instructions that can be employed to program a computer or other processor to implement various aspects of the present invention as discussed above. Additionally, it should be appreciated that according to one aspect of this embodiment, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, the data record may be stored in computer-readable media in any suitable form. For simplicity of illustration, data records may be shown to have data record entries. However, any suitable mechanism may be used to establish locations within the data record to store data, such as parameter values.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the invention may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Further, some actions are described as taken by a “user” or “patient”. It should be appreciated that a “user” or “patient” need not be a single individual, and that in some embodiments, actions attributable to a “user” or “patient” may be performed by a team of individuals and/or an individual in combination with computer-assisted tools or other mechanisms.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

The present invention is defined by the following independent claims. Preferred embodiments are described by the following dependent claims as well as by aspects, examples and embodiments described above.

What is claimed is:

1. A method of compression therapy implemented on a computing device, said method comprising:
using at least one processor to perform the following operations:
obtaining an identifier of a compression therapy arrangement, the compression therapy arrangement including a compression garment;
determining an operational state of a portion of the compression garment based on the obtained identifier; and
generating instructions for a perceptible representation of an indicator of the determined operational state.

2. The computing device-implemented method of claim 1, the operations further comprising:
based on the obtained identifier, obtaining at least one range of allowable and/or targeted values for at least one operational parameter of the portion of the compression garment,
wherein the indicator indicates the obtained at least one range of allowable and/or targeted values,
wherein the determining of the operational state includes obtaining one or more measured values for the at least one operational parameter of the portion of the compression garment, and
wherein the determining of the operational state includes comparing the one or more measured values with the at least one range of allowable and/or targeted values, and wherein the indicator indicates a result of the comparing of the one or more measured values with the at least one range of allowable or targeted values.

3. The computing device-implemented method of claim 1, the operations further comprising:
scanning a code, wherein the code encodes the identifier of the compression therapy arrangement and wherein the identifier is obtained through the scanning of the code.

4. The computing device-implemented method of claim 2,
wherein the indicator indicates how the measured values relate to the range of allowable or targeted values and indicates the at least one operational parameter and further indicates a maximum and a minimum of allowable or targeted values of the range of allowable and targeted values and includes a graphical illustration of a portion of a human body, and
wherein the indicator indicates how the measured values relate to the range of allowable and targeted values at a location of a graphical user interface that is associated with a subpart of the graphical illustration corresponding to a body portion at which the portion of the compression garment is applied to a patient.

5. The computing device-implemented method of claim 2,
wherein the at least one operational parameter includes an external pressure exerted by the compression garment on a body portion of a patient and an operating time or wearing time of the portion of the compression garment, and/or wherein the at least one operational parameter includes a number of washing cycles the compression garment has experienced.

6. The computing device-implemented method of claim 4,
wherein the portion of the compression garment includes a plurality of subparts of the compression garment, the at least one range of allowable or targeted values includes a plurality of ranges of allowable or targeted values for the at least one operational parameter with each range of the plurality of ranges being associated with a corresponding subpart of the plurality of subparts, and wherein the indicator indicates, for each subpart of at least some of the plurality of subparts of the compression garment, how respective ones of the measured values measured for the respective subpart relate to an associated one of the ranges of allowable and/or targeted values corresponding to the subpart of the plurality of subparts.

7. The computing device-implemented method of claim 6, the operations further comprising:
receiving, for a subpart of the plurality of subparts or the portion of the compression garment, a change of a setting corresponding to the at least one operational parameter; and
in response to the receiving of the change of the setting, generating instructions for a perceptible representation of a feedback message indicating whether or not the received change of the setting is in accordance with the at least one range of allowable or targeted values that is associated with the subpart of the portion of the compression garment.

8. The computing device-implemented method of claim 6, wherein the operational state includes an operational state of a particular subpart of the plurality of subparts, wherein the indicator indicates that the particular subpart is not yet activated for read-out of the at least one operational parameter;
wherein the method further comprises:
receiving, by the computing device, an activation signal for activating read-out of the at least one operational parameter for the particular subpart; and
in response to the receiving of the activation signal, changing the indicator of the operational state of the particular subpart such that the changed indicator indicates that the particular subpart is activated for read-out of the at least one operational parameter,
wherein the at least one range of allowable and/or targeted values is obtained from a database via a network connection.

9. The computing device-implemented method of claim 1, wherein the operations further comprise:
storing the obtained measured values on a mobile communication device and transmitting, via a network connection, the obtained measured values to a pre-approved third party server;
generating, by the processor, instructions for generating a perceptible representation of guiding information, the guiding information including instructions for a user how to properly operate the compression garment;
generating instructions for a perceptible representation of behavior information, the behavior information including instructions for a user how to behave to comply with a particular compression therapy;
transmitting, for each subpart of the plurality of subparts of the compression garment or for the portion of the compression garment the corresponding measured values and associated timestamps to a database for storage in the database; and
receiving, from a database, based on historical values stored in the database wherein the historical values include values previously measured for the at least one operational parameter and associated historical timestamps, instructions for generating a perceptible representation of the recommendation information to a user, the recommendation information including behavioral recommendations for a user to comply with a particular compression therapy and recommendations regarding proper usage of the compression garment.

10. A compression therapy arrangement comprising:
a compression garment adapted to exert external pressure on a body portion;
a mobile communication device, wherein the mobile communication device has a processor operable to process therapy signals;
at least one sensor device associated with the compression garment and having at least one pressure sensor capable of producing at least one pressure signal indicative of the external pressure;
a transmitter; wherein the transmitter is linked to the at least one sensor device and operable to transmit therapy signals corresponding to the pressure signals to the mobile communication device; and
an identifier of the compression garment, wherein the identifier is encoded by a code,
wherein the code is scannable such that the identifier is obtainable through scanning of the code, and
wherein the processor is operable to execute the operations according to claim 1.

11. The compression therapy arrangement according to claim 10,
wherein the at least one pressure sensor comprises at least one capacitor having a capacity which is dependent on the external pressure, and
wherein the at least one capacitor is integrated into an electric circuit further comprising a resistance and an inductance, wherein the pressure signal is determined based on a frequency analysis.

12. The compression therapy arrangement of claim 10,
wherein the at least one sensor device further comprises at least a bioimpedance sensor system, a movement sensor, an acceleration sensor, and/or a position sensor, and
wherein the transmitter is operable to transmit to a mobile communication device at least a movement signal derived from the movement sensor, an acceleration signal derived from the acceleration sensor, and a position signal derived from the position sensor, and wherein the signal is transmitted as digital data.

13. The compression therapy arrangement according to claim 10, further comprising:
a storage means;
a converter, linked to said processor means and operable to generate a perceptible representation of a signal, wherein the converter is linked to the processor and operable to generate a perceptible signal; and
and an input means, wherein the input means is linked to the processor and is operable to input external data,
wherein the processor is operable to compare input target data with progress data derived from the stored pressure data and/or stored activity data,
wherein the processor is, in particular, further operable to receive web data from an external web server and to control the converter to generate a perceptible representation of the information content of the web data together with a graphic representation of the pressure data and/or the external data,
wherein the processor is, in particular, further operable to transmit pressure data, user data, device data, and/or target data to an external web server,
wherein the processor is further operable to receive web data from an external web server and to control the converter to generate a perceptible representation of the information content of the web data together with a perceptible representation of the pressure data and/or the external data,
wherein the processor is further operable to receive usage data indicative of the period of usage of the compression garment and to compare the usage data to respective target data,
wherein the processor is further operable to transmit data to one or more external hosts, and
wherein the processor is further operable to divide and/or encrypt data for transmission to the at least one external host.

14. The compression therapy arrangement according to claim 10,
wherein the compression garment comprises at least one compression sleeve, one compression bandage, and/or at least one adjustable compression strap; and
further comprising at least two spaced apart pressure sensors, each spaced apart pressure sensor being associated with a corresponding subpart of the compression garment, one spaced apart pressure sensor being arranged in a distal portion of the compression garment, and the other spaced apart pressure sensor being arranged in a proximal portion of the compression garment.

15. The compression therapy arrangement according to claim 10,
wherein the code is a QR code or a bar code, and wherein the code is attached to the compression garment.
